(19) United States
(12) Patent Application Publication
(10) Pub. No.: US 2023/0149626 A1
(43) Pub. Date: May 18, 2023

(54) INTRAVENOUS SET DURATION MONITOR

(71) Applicant: CareFusion 303, Inc., San Diego, CA (US)

(72) Inventors: Shawn Wayne Dekalb, Prescott, AZ (US); Jeffrey Wen-Yu Chen, San Diego, CA (US)

(21) Appl. No.: 17/955,981

(22) Filed: Sep. 29, 2022

Related U.S. Application Data
(60) Provisional application No. 63/280,421, filed on Nov. 17, 2021.

(51) Int. Cl. A61M 5/168; G06K 7/10

(52) U.S. Cl. CPC A61M 5/168; G06K 7/10297; A61M 2205/584; A61M 2205/60; A61M 2205/3327

(57) ABSTRACT

An IV set includes an IV component and a duration monitor device coupled to the IV component. The duration monitor device may include an indicator portion configured to change color after a determined amount of time upon activation of the indicator portion, or an RFID tag configured to be detected by an RFID reader. The duration monitor is configured to provide an indication of passage of a useful-life time period for the IV set or the IV component. Methods of operating a duration monitor device are also provided.

### CROSS-REFERENCE TO RELATED APPLICATIONS

This application claims the benefit of priority under 35 U.S.C. § 119 to U.S. Provisional Patent Application Ser. No. 63/280,421, entitled “INTRAVENOUS SET DURATION MONITOR,” filed on Nov. 17, 2021, the entirety of which is incorporated herein by reference.

### TECHNICAL FIELD

The present disclosure generally relates to intravenous (IV) set devices, in particular IV set duration monitors.

### BACKGROUND

Typical infusion or intravenous (IV) sets are constructed by joining multiple translucent polymeric tubing segments to multiple polymeric components, many of which are also translucent. These IV sets are then used with infusion pumps or gravity systems to provide fluids to a user, such as a patient. However, an IV set typically has both a predetermined shelf life (e.g., 3-5 years storage time) and a predetermined use-life (e.g., 24 hours, 72 hours, 96 hours, 7 days). In particular, once an IV set is removed from its packaging and put into use, the IV set should only be used for a length of time up to the use-life, after which time the IV set should be changed out. The time of use may be determined by healthcare regulations, the type(s) of drugs, medications or substances that are being infused through the IV set, and the types of materials used to make the IV set. When an IV set is used beyond its intended life, components can fatigue, durability of the IV set can fail, and special components may no longer function as needed, such as the function of a filter or a clamp.

Currently, there is no good way to alert the clinician when an IV set should or needs to be changed. Typically, when the IV set is put into use, it is up to the clinician to chart or note the start time of the IV set use. However, in the hectic environment of some medical settings, the clinician may not have time to chart or note the start time. In addition, it may not be clear which use-life time period a particular IV set in use requires. Further, when a clinician does determine that an IV set change is required, the action of changing the IV set is time and labor intensive, requiring locating the set, determining the medication being provided by the set and manually labeling IV lines. If the patient has several IV lines, such as in the intensive care unit (ICU), the task of finding and changing out the correct IV set(s) can be time consuming.

For these reasons, it is desirable to provide devices and methods for providing IV sets with a duration monitor that alerts when the associated IV set should be replaced, thus giving a clinician an indication to change the IV set.

### SUMMARY

In one or more embodiments according to the disclosure, an IV set comprises an IV component and a duration monitor device coupled to the IV component, the duration monitor device comprising an indicator portion configured to change color after a determined amount of time upon activation of the indicator portion, wherein the duration monitor is configured to provide an indication of passage of a use-life time period of one of the IV set and the IV component.

In one or more embodiments according to the disclosure, an IV set comprises an IV component and a duration monitor device coupled to the IV component, the duration monitor device comprising an RFID tag configured to be detected by an RFID reader, wherein the duration monitor is configured to provide an indication of passage of a use-life time period of one of the IV set and the IV component upon being detected by the RFID reader.

In one or more embodiments according to the disclosure, a method of operating a duration monitor device, comprises: coupling the duration monitor device to an intravenous (IV) set component; activating the duration monitor device upon placing the IV set component into service; detecting a change in color of an indicator portion of the duration monitor device; determining a passage of a use-life time period of one of the IV set component and an IV set comprising the IV set component based on the detected change in color of the indicator portion; and remove the one of the IV set component and the IV set comprising the IV set component from service.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIGS. 2A-2C depict top views of example IV sets and extension sets.

FIG. 3 depicts a front view of an IV set duration monitor, according to aspects of the disclosure.

FIG. 4 depicts a perspective view of an IV set component with a duration monitor, according to aspects of the disclosure.

FIG. 5 depicts a perspective view of an IV set with a duration monitor, according to aspects of the disclosure.

FIG. 6 depicts a perspective view of an IV set having a component with a duration monitor, according to aspects of the disclosure.

FIG. 7 depicts a front view of an IV set duration monitor, according to aspects of the disclosure.

FIG. 8 depicts a perspective view of an IV set component with a duration monitor, according to aspects of the disclosure.

FIG. 9 illustrates a method of making an IV set with a duration monitor device, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

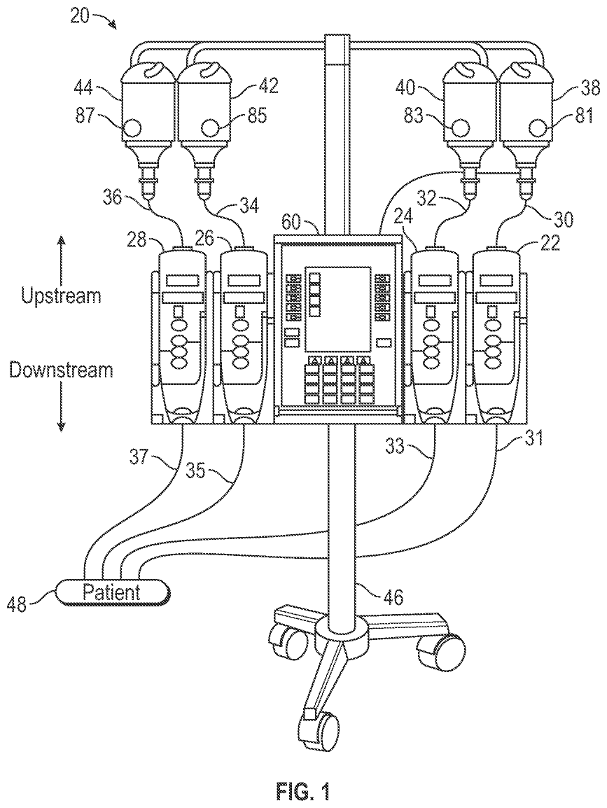

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

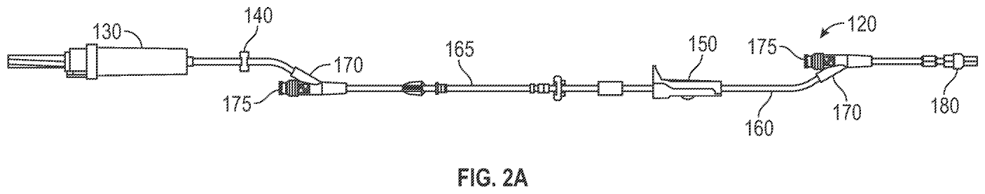

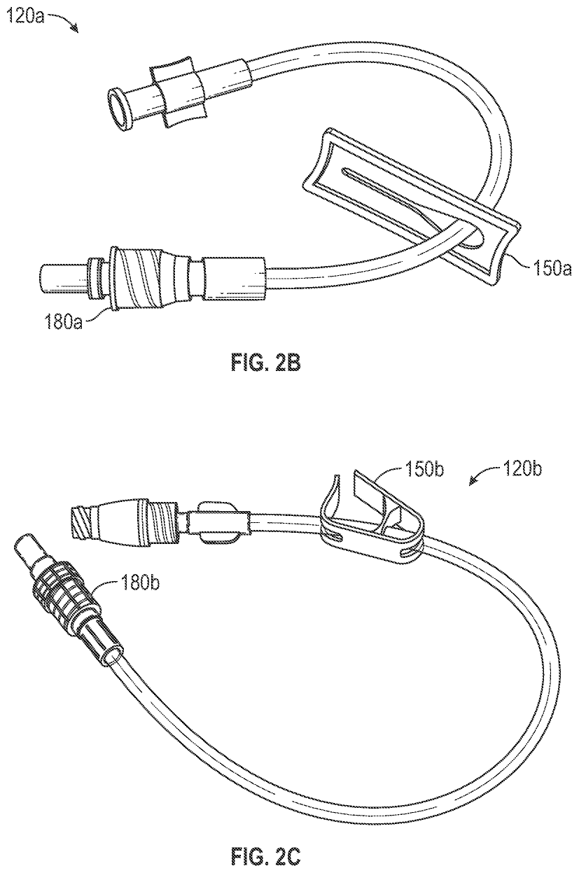

Typically, medical fluid administration sets have more parts than are shown in FIG. 1, such as those shown in FIGS. 2A-2C. Infusion sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIGS. 2A, an infusion set 120 may include a drip chamber 130, a check valve 140, a flow controller 150 (e.g., roller clamp) and a pump segment 165 connected together by tubing 160. The infusion set 120 may also include a Y-site 170 having a Y-shaped junction with a needleless port 175, as well as a luer lock connector 180 at the end of the infusion set 120. The luer lock connector 180 may be used for connection to a catheter inserted into a patient, for example. The infusion set 120 may include additional infusion components and may be formed of any combination of components and the tubing 160. For example, substituting a length of tubing 160 for the pump segment 165 of infusion set 120 can change the infusion set 120 from a pump IV set to a gravity IV set.

As shown in FIG. 2B, an IV extension set 120a may be used to connect any two infusion components or devices, such as IV sets, infusion pumps, syringe pumps and the like. IV extension set 120a includes a luer lock connector 180a and a flow controller 150a. Similarly, as shown in FIG. 2C, another IV extension set 120b includes a luer lock connector 180b and a flow controller 150b.

In aspects of the disclosure, the subject technology provides a duration monitor as part of the IV set. For example, the IV set may have a built-in duration monitor that visually shows the time of use of the IV set. This provides for accurate use of IV sets, eliminating both too frequent changing of IV sets (e.g., clinician not sure when IV set use-life is up and changes IV set too frequently, wasting the remaining use-life hours of the replaced IV set) and overuse of an IV set past the use-life of the IV set (e.g., clinician not sure when IV set use-life is up and changes IV set too infrequently, possibly causing medical, administrative and legal issues or IV set/component failure).

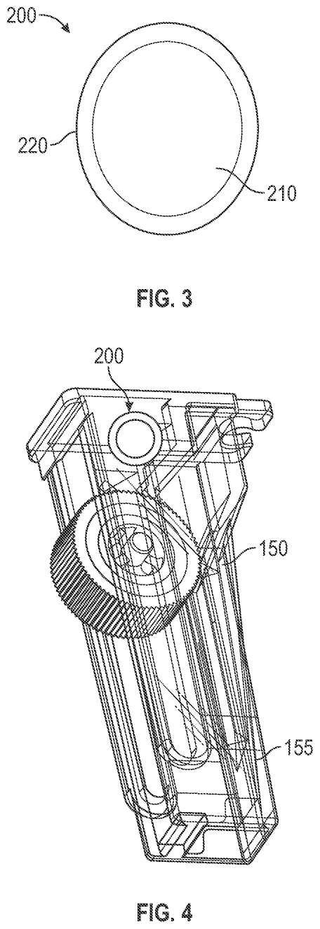

As shown in FIG. 3, a duration monitor device 200 is provided. In aspects of the disclosure, duration monitor device 200 may be a label or a tag having a structure that changes color when exposed to a predetermined amount of humidity, such as humidity in the ambient air where the IV set is being used. For example, the duration monitor device 200 may include one or more chemicals (e.g., cobalt chloride, cobalt free chloride, special plastics) that cause the duration monitor device 200 to be initiated when the IV set of which the duration monitor device 200 is associated is removed from its packaging (e.g., vacuum sealed) and exposed to humidity in the air. As another example, the duration monitor device 200 may be initiated when a cover is removed (e.g., peeled off), again exposing the duration monitor device 200 to humidity in the air.

In aspects of the disclosure, duration monitor device 200 may have a structure that changes color when exposed to light (e.g., fluorescent, incandescent, halogen, LED). For example, the duration monitor device 200 may include one or more chemicals (e.g., liquid crystal polymer, modified poly(acrylamides)s) that cause the duration monitor device 200 to be initiated when the IV set of which the duration monitor device 200 is associated is removed from its packaging (e.g., vacuum sealed) and exposed to light in the room. As another example, the duration monitor device 200 may be initiated when a cover is removed (e.g., peeled off), again exposing the duration monitor device 200 to light. As yet another example, the duration monitor device 200 may be initiated when exposed to a specific wavelength of light (e.g., ultraviolet (UV), infrared (IR)). Here, the once the duration monitor device 200 is removed from its packaging or a cover is peeled off, a particular light source (e.g., UV, IR) may be shone on the exposed duration monitor device 200 to activate or initiate it.

As shown in FIG. 3, the duration monitor device 200 includes an indicator portion 210 that is disposed on a backing portion 220. The backing portion 220 may have an adhesive coating so that the duration monitor device 200 may be attached or affixed to an IV component, such as the flow controller 150 shown in FIG. 4. The indicator portion 210 as shown in FIG. 3 is the center circle portion, which changes color when it reaches the use-life of the IV set, whereas the visible portion of the backing portion 220 is the perimeter surrounding the indicator portion 210, which does not change color. In aspects of the disclosure, the indicator portion 210 may be any portion of the visible surface of the duration monitor device 200 (e.g., the entire surface). In storage and prior to use, the indicator portion 210 may have a storage bag or a peel off label covering it and protecting the indicator portion 210 from exposure to humidity in the air or light. Accordingly, when the IV set is put into use, the storage bag or peel off label may be removed and the subsequent exposure to humidity in the air or light starts a timer function of the indicator portion 210.

In aspects of the disclosure, the chemical structure or makeup of the indicator portion 210 may be configured to change color once the predetermined use-life time period (e.g., 72 hours) has passed. Thus, the initial color of the indicator portion 210 (e.g., white, green) may stay the same from its inactivated (e.g., stored) state, its activation or initiation (e.g., exposure to humidity or light) and through 72 hours of use after activation/initiation. After the 72 hours have passed, the indicator portion 210 may change color (e.g., yellow, red) to visually indicate the associated IV set or IV component has reached its use-life and needs to be replaced.

Thus, with a quick glance at the indicator portion 210 of a duration monitor device 200 for an IV set that is in use, a clinician can easily and quickly determine if the associated IV set component or IV set has reached its use-life maximum. For example, if the indicator portion 210 shown in FIG. 3 is a first color (e.g., green), the clinician can quickly determine that the associated IV set component or IV set is within its rated use-life and leave the IV set component or IV set in use. Similarly, if the indicator portion 210 shown in FIG. 3 is a second color (e.g., yellow), the clinician can quickly determine that the associated IV set component or IV set has reached its rated use-life and change out the used IV set component or IV set with a new one.

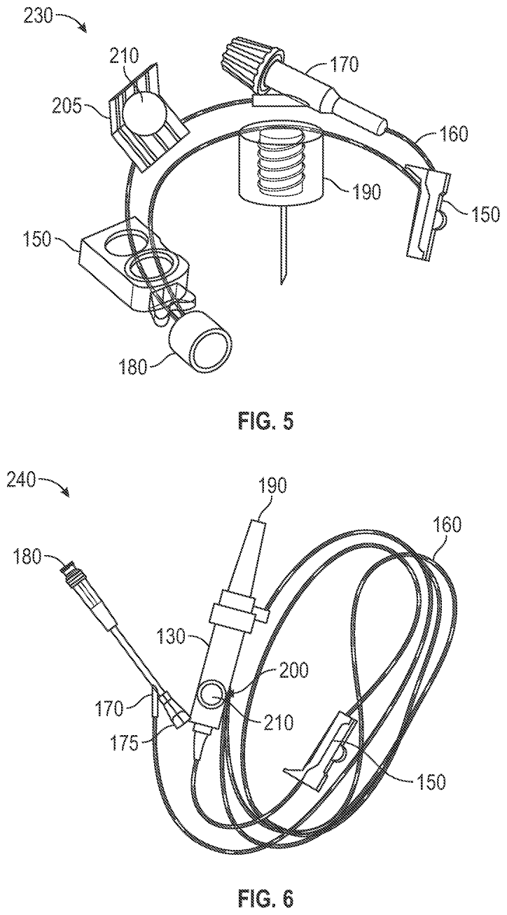

In aspects of the disclosure, a duration monitor device may have any suitable form or format. For example, instead of the circular form shown in duration monitor device 200, the duration monitor device may be a linear strip patch, an oval patch, a square patch or any other geometric shaped patch where the indicator portion changes color at the rated use-life time. Here, any number of duration monitor devices 200 may be applied to one or more IV set components, such as a flow controller 150 as shown in FIG. 4, as a label 205 attached to IV tubing 160 as shown in FIG. 5, and/or a drip chamber 130 as shown in FIG. 6, for example.

In aspects of the disclosure, a duration monitor device may be an integral portion of an IV set component instead of an externally applied tag or patch. For example, the duration monitor device 200 of the flow controller 150 shown in FIG. 4 may be an indicator portion 210 of color changing material that is integrally formed with the housing 155 of the flow controller 150 (e.g., co-injected, co-molded, over-molded, coated). Here, the indicator portion 210 of color changing material functions similarly to the indicator portion 210 of duration monitor device 200 described above, where the indicator portion 210 is activated when removed from a sealed package or a cover is peeled off and the indicator portion 210 is exposed to humidity or light. Here, the rest of the housing 155 of flow controller 150 may maintain its initial color, while only the indicator portion 210 of the flow controller housing 155 may change color when the flow controller 150 and/or the IV set that the flow controller 150 is part of reaches the maximum use-life time period.

In aspects of the disclosure, a duration monitor device may be an entire housing or body of an IV set component. For example, referring again to the flow controller 150 of FIG. 4, the entire housing 155 may be formed of or be coated with a color changing material instead of just the indicator portion 210 shown. Again, the color changing material may be co-injected or co-molded along with any other suitable materials to form the housing 155, or the color changing material may be over-molded or coated onto a base material of the housing 155. Here, the entire flow controller housing 155 may change color when the flow controller 150 and/or the IV set that the flow controller 150 is part of reaches the maximum use-life time period.

FIG. 5 shows an example IV extension set 230 having a combination of infusion components and IV tubing 160. For example, extension set 230 includes a Y-site 170, a spike 190, a luer lock connector 180, IV tubing 160 and flow controllers 150. A duration monitor device 200 is affixed to the IV tubing 160 via a label 205, the duration monitor device 200 having an indicator portion 210 that changes color after a set time (e.g., use-life time) once it is activated. Here, the duration monitor device 200 may be affixed to the IV tubing 160 via the label 205 if there are no infusion components within the IV extension set 230 that are suitable (e.g., not large enough, will be partially or totally obscured during use) to include the duration monitor device 200.

According to aspects of the disclosure, when the IV extension set 230 is removed from vacuum sealed or airtight packaging, the indicator portion 210 may be automatically activated upon exposure to humidity in the ambient air or light and start its timing function. According to aspects of the disclosure, when the IV extension set 230 is put in use, a peel off label or covering may be peeled off (e.g., removed) from the indicator portion 210, which then may be activated upon exposure to humidity or light and start its timing function.

FIG. 6 shows an example IV set 240 that includes a drip chamber 130 with a spike 190, a flow controller 150, a Y-site 170 with a needless port 175, a luer lock connector 180 and IV tubing 160. IV set 240 includes a duration monitor device 200 disposed on the drip chamber 130 as a label and having an indicator portion 210 that changes color over time after being activated. Here, the duration monitor device 200 may be disposed on the drip chamber 130 because the drip chamber 130 is suitable (e.g., large enough, is most visible during use) to include the IV set timer device 200.

In aspects of the disclosure, a duration monitor device may be integral to an IV set component, as discussed above. For example, the duration monitor device 200 shown in FIG. 6 may be formed as a section of the drip chamber 130 itself and/or the entire see through body of the drip chamber 130 may be include a duration monitor device material that changes the color of the body over time while maintaining visibility into the drip chamber 130. Similarly, a section of IV tubing 160 or an entire length of IV tubing 160 may include a duration monitor device material that changes the color of the section of IV tubing 160 or the entire length of IV tubing 160 over time while maintaining visibility into the IV tubing 160.

In aspects of the disclosure, any IV set component may include an integrally formed portion of duration monitor device material, such as drip chamber 130, check valve 140, flow controller 150, tubing 160, Y-site 170, luer lock connector 180, catheter 185, spike 190, and the like. In any of the above, the duration monitor material portion of the IV set component may be activated by peeling off a cover or label, by being removed from airtight or vacuum sealed packaging, by being exposed internally and/or externally to a conductive fluid (e.g., saline, anesthetics, nutritions) or any other suitable activation process.

In aspects of the disclosure, the duration monitor device 200 may be added to an IV set component during manufacturing and packaging of the IV set. In aspects of the disclosure, the duration monitor device 200 may be provided separately on a roll, a sheet, in individual packages or any other suitable storage device, where the clinician or user applies the duration monitor device 200 to some portion of the IV set when the IV set is put into use.

For example, a roll or sheet of duration monitor devices 200 with cover labels may be on hand in the medical treatment area. Here, when a new IV set or even a new single IV set component is put into use, the clinician removes a duration monitor device 200 from the roll or sheet, thus exposing an adhesive back surface, and applies the adhesive surface to an IV set component, then peeling off a front cover label to activate the duration monitor device 200.

As another example, the duration monitor device 200 may be sealed in an airtight or vacuum sealed individual package. Here, when a new IV set or new single IV set component is put into use, the clinician removes a duration monitor device 200 from the sealed individual package, exposing the indicator portion 210 to the elements (e.g., humidity, light) to activate the duration monitor device 200, peels off a cover to expose an adhesive back surface, and applies the adhesive surface to an IV set component

In aspects of the disclosure, the duration monitor device 200 may be applied by an applicator device (e.g., applicator gun). For example, a handheld applicator device (e.g., like a tape gun or label applicator) may be used by the clinician to apply duration monitor devices 200 to IV set components as needed. Here, the applicator device may lift the duration monitor device 200 from a backing roll to expose an adhesive surface of the duration monitor device 200, and peel off a cover label from the indicator portion 210 to activate the duration monitor device 200 upon application from the applicator device to the IV set component. As another example, the applicator device may be a robotic or machine portion of a manufacturing system or assembly system, where the duration monitor device 200 is automatically applied to an IV set component during manufacturing, assembly or packaging of an IV set.

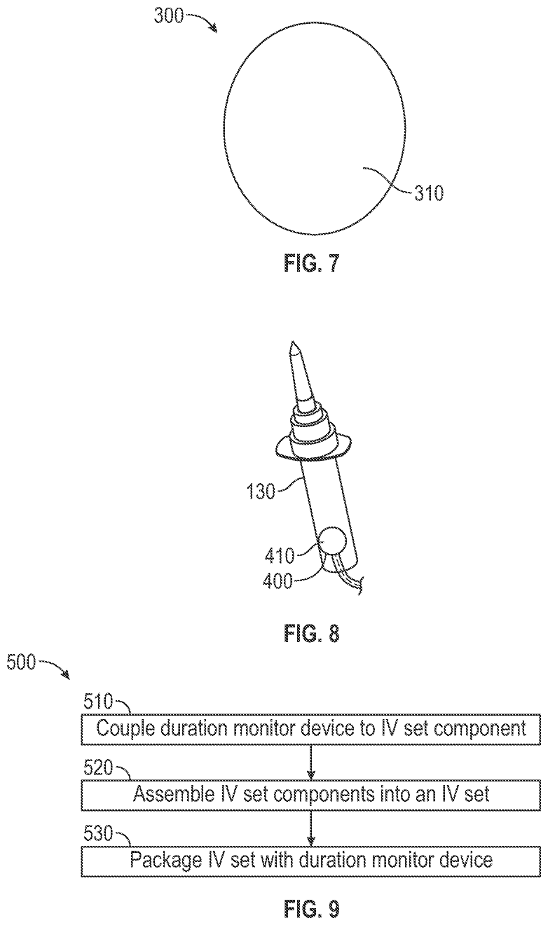

As shown in FIG. 7, a duration monitor device 300 is provided. In aspects of the disclosure, duration monitor device 300 may be a passive radio frequency identification (RFID) tag 310 having a structure that can be read by an RFID reader where the IV set (e.g., IV set 120) is being used. For example, the RFID tag 310 may be detected by an RFID reader when the IV set of which the duration monitor device 300 is associated is removed from its RFID shielded packaging. As another example, the RFID tag 310 may be detected when the IV set of which the duration monitor device 300 is associated is installed or put into service within a range of an RFID reader. For example, an RFID reader may be a remote proximity sensor on a pump system or a cabinet.

In aspects of the disclosure, an RFID reader may be part of an infusion pump system (e.g., patient care system 20), such that when the RFID tag 310 is placed within a specific distance of the infusion pump system, the RFID tag 310 is recognized by the RFID reader and an IV set initiation may be activated. For example, a query may be generated by the infusion pump system for a user (e.g., clinician) to accept initiation of the IV set, to start a log of the IV set and/or to enable an alarm process. Here, the alarm process may include using a timer to monitor the amount of time that passes after initiation of the IV set and triggering an alarm (e.g., visual indicator, light, horn, text message) after a predetermined use-life time period has passed (e.g., 72 hours).

In aspects of the disclosure, a duration monitor device 300 may have any suitable form or format. For example, instead of the circular form shown in duration monitor device 300, the duration monitor device may be a linear strip RFID tag, a rectangular RFID tag, a square RFID tag or any other geometric shaped RFID tag where the passive RFID tag may be read by an RFID reader to initiate and monitor the rated use-life time of the IV set component or IV set associated with the RFID tag 310. Here, any number of duration monitor devices 300 may be applied to one or more IV set components, such as in place of or in addition to duration monitor device 200 on flow controller 150 shown in FIG. 4, duration monitor device 200 on label 205 attached to IV tubing 160 shown in FIG. 5, and/or duration monitor device 200 on drip chamber 130 shown in FIG. 6, for example. For example, a duration monitor device 200 on a flow controller 150 may be activated when the IV set is removed from sealed packaging, while a duration monitor device 300 on a drip chamber 130 may be read when the IV set is removed from the sealed packaging and the IV set is placed within range of an RFID reader. Here, the duration monitor device 200 may change color when the use-life time period is reached, while an infusion pump system having an RFID reader that detected duration monitor device 300 may trigger an alarm when the use-life time period is reached.

As shown in FIG. 8, a duration monitor device 400 is provided. In aspects of the disclosure, duration monitor device 400 may be a radio frequency identification (RFID) tag 410 having open circuitry that requires contact with a conductive fluid (e.g., saline, anesthetics, nutritions) for the circuit to be completed and read by an RFID reader where the IV set (e.g., IV set 120) is being used. For example, the RFID tag 410 may be detected by an RFID reader when the IV set of which the duration monitor device 400 is associated is put into use within a range of an RFID reader and a conductive IV fluid flows through the IV set component (e.g., drip chamber 130). I

In aspects of the disclosure, the RFID tag 410 may be disposed within the drip chamber 130 such that exposed moisture contact (e.g., saline flow) with a resistive material of the RFID tag 410 establishes electrical connectivity and completes a circuit in the RFID tag 410, which then can be read by the RFID reader. In aspects of the disclosure, the RFID tag 410 may be disposed on the exterior of the drip chamber 130 with electrical contacts disposed within the drip chamber 130 such that exposed moisture contact (e.g., saline flow) with the internally disposed contacts of the RFID tag 410 establishes electrical connectivity and completes a circuit in the RFID tag 410, which then can be read by the RFID reader. In aspects of the disclosure, the RFID tag 410 may be disposed on the exterior of the drip chamber 130 such that exposed moisture contact (e.g., wet by a user) with a resistive material of the RFID tag 410 establishes electrical connectivity and completes a circuit in the RFID tag 410, which then can be read by the RFID reader.

In aspects of the disclosure, the RFID tag 410 may be disposed on or within any IV set component. For example, any number of duration monitor devices 400 may be applied to one or more IV set components, such as in place of or in addition to duration monitor device 200 on flow controller 150 shown in FIG. 4, duration monitor device 200 on label 205 attached to IV tubing 160 shown in FIG. 5, and/or duration monitor device 200 on drip chamber 130 shown in FIG. 6, for example. For example, a duration monitor device 200 on a flow controller 150 may be activated when the IV set is removed from sealed packaging, while a duration monitor device 400 on a drip chamber 130 may be activated when a saline fluid begins running through the drip chamber 130. Here, the duration monitor device 200 may change color when the use-life time period is reached, while an infusion pump system having an RFID reader that detected duration monitor device 400 may trigger an alarm when the use-life time period is reached.

In aspects of the disclosure, an RFID reader may be part of an infusion pump system (e.g., patient care system 20), such that when the RFID tag 410 is placed within a specific distance of the infusion pump system and a circuit of the RFID tag 410 is completed by exposed moisture contact, the RFID tag 410 is recognized by the RFID reader and an IV set initiation may be activated. For example, a query may be generated by the infusion pump system for a user (e.g., clinician) to accept initiation of the IV set, to start a log of the IV set and/or to enable an alarm process. Here, the alarm process may include using a timer to monitor the amount of time that passes after initiation of the IV set and triggering an alarm (e.g., visual indicator, light, horn, text message) after a predetermined use-life time period has passed (e.g., 72 hours).

According to some aspects of the disclosure, a method 500 of making an IV set with a duration monitor device is shown in FIG. 9. In step 510, a duration monitor device (e.g., duration monitor device 200, 300, 400) is coupled with (e.g., disposed on or within, formed integrally with, attached to) an IV set component (e.g., drip chamber 130, flow controller 150, IV tubing 160). In aspects of the disclosure, the duration monitor device may be an adhesive label or tag that is attached to (e.g., stuck to) the IV set component. In aspects of the disclosure, the duration monitor device may be integrally formed with the IV set component. In aspects of the disclosure, the duration monitor device may include a color changing material and/or an RFID circuit.

An IV set is assembled in step 520. For example, various IV set components may be assembled into a complete IV set (e.g., IV extension set 230, IV set 240). In aspects of the disclosure, the IV set is assembled first in step 520 and then the duration monitor device is added to the IV set component. In aspects of the disclosure, the duration monitor is added to the IV set component and then the IV set is assembled.

In step 530, the IV set with duration monitor device is packaged. For example, the IV set with duration monitor device may be inserted into an airtight bag and the air in the bag is extracted. As another example, the IV set with duration monitor may be vacuum sealed into a bag or other packaging form. In yet another example, the IV set with duration monitor device may be inserted into RFID shielded bag or package.

In some embodiments according to the disclosure, an IV set comprises an IV component and a duration monitor device coupled to the IV component, the duration monitor device comprising an indicator portion configured to change color after a determined amount of time upon activation of the indicator portion, wherein the duration monitor is configured to provide an indication of passage of a use-life time period of one of the IV set and the IV component.

In aspects of the disclosure, the indicator portion comprises one or more chemicals configured to activate when exposed to humidity in ambient air. In aspects of the disclosure, the indicator portion is exposed to the humidity upon one of opening sealed packaging containing the IV set and removing the IV set from sealed packaging. In aspects of the disclosure, the duration monitor device further comprises a cover disposed on the indicator portion, wherein the indicator portion is exposed to the humidity upon removal of the cover from the indicator portion.

In aspects of the disclosure, the indicator portion comprises one or more chemicals configured to activate when exposed to a light source. In aspects of the disclosure, the indicator portion is configured to be exposed to the light source upon removing the IV set from light blocking packaging. In aspects of the disclosure, the duration monitor device further comprises a cover disposed on the indicator portion, wherein the indicator portion is configured to be exposed to the light source upon removal of the cover from the indicator portion. In aspects of the disclosure, the indicator portion is configured to be activated only by exposure to a specific wavelength of light.

In aspects of the disclosure, the duration monitor device comprises a backing portion and the indicator portion is disposed on the backing portion. In aspects of the disclosure, the indicator portion is disposed on a first side of the backing portion, and wherein an adhesive coating is disposed on a second side of the backing portion, the adhesive coating configured to one of affix the duration monitor device to a surface of the IV component, affix the duration monitor device to a tag coupled to the IV component and affix a first portion of the backing portion to a second portion of the backing portion.

In aspects of the disclosure, the duration monitor device is a portion of material integrally formed with the IV component, and wherein upon exposure of the IV component to an activation source, the remaining portion of the IV component is configured to maintain its color over time and an integrally formed indicator portion is configured to change color after the determined amount of time. In aspects of the disclosure, the duration monitor device comprises a first material integrally formed with a second material of a body of the IV component by one of co-injection and co-molding, wherein the body of the IV component is configured to change color after the determined amount of time upon the activation. In aspects of the disclosure, the duration monitor device comprises an indicator material disposed on a base material of a body of the IV component by one of coating and over-molding, wherein the body of the IV component is configured to change color after the determined amount of time upon the activation.

In some embodiments according to the disclosure, an IV set comprises an IV component and a duration monitor device coupled to the IV component, the duration monitor device comprising an RFID tag configured to be detected by an RFID reader, wherein the duration monitor is configured to provide an indication of passage of a use-life time period of one of the IV set and the IV component upon being detected by the RFID reader.

In aspects of the disclosure, the RFID tag is configured to be exposed to the RFID reader upon removing the IV set from RFID shielded packaging. In aspects of the disclosure, the RFID tag is configured to be detected by the RFID reader upon being moved within a proximity range of an infusion pump system.

In some embodiments according to the disclosure, a method of operating a duration monitor device, comprises: coupling the duration monitor device to an intravenous (IV) set component; activating the duration monitor device upon placing the IV set component into service; detecting a change in color of an indicator portion of the duration monitor device; determining a passage of a use-life time period of one of the IV set component and an IV set comprising the IV set component based on the detected change in color of the indicator portion; and remove the one of the IV set component and the IV set comprising the IV set component from service.

In aspects of the disclosure, the indicator portion of the duration monitor device comprises one or more chemicals configured to activate when one of being exposed to humidity in ambient air and being exposed to a light source, the method further comprising one of: activating the duration monitor device by removing the IV set component from protective packaging; and activating the duration monitor device by removing a cover removably disposed on the indicator portion.

In aspects of the disclosure, the duration monitor device is coupled to the IV set component upon placing the IV set component into service, the method further comprising one of: removing the duration monitor device from one of a roll and a sheet and placing an adhesive surface of the duration monitor device on a portion of the IV set component, then removing a cover from the indicator portion to activate the duration monitor device; removing the duration monitor device from a sealed package and placing an adhesive surface of the duration monitor device on a portion of the IV set component, wherein an uncovered indicator portion is activated upon removal of the duration monitor device from the package; and applying the duration monitor device by an applicator device to place an adhesive surface of the duration monitor device on a portion of the IV set component and to remove a cover from the indicator portion to activate the duration monitor device.

In aspects of the disclosure, coupling the duration monitor device to the IV set component comprises one of: co-injecting an indicator material and a base material to form a body of the IV set component; co-molding an indicator material and a base material to form a body of the IV set component; coating an indicator material on a base material of a body of the IV set component; and over-molding an indicator material onto a base material of a body of the IV set component.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) set, comprising:
an IV component; and
a duration monitor device coupled to the IV component, the duration monitor device comprising an indicator portion configured to change color after a determined amount of time upon activation of the indicator portion,
wherein the duration monitor is configured to provide an indication of passage of a use-life time period of one of the IV set and the IV component.

2. The IV set of claim 1, wherein the indicator portion comprises one or more chemicals configured to activate when exposed to humidity in ambient air.

3. The IV set of claim 2, wherein the indicator portion is exposed to the humidity upon one of opening sealed packaging containing the IV set and removing the IV set from sealed packaging.

4. The IV set of claim 2, the duration monitor device further comprising a cover disposed on the indicator portion, wherein the indicator portion is exposed to the humidity upon removal of the cover from the indicator portion.

5. The IV set of claim 1, wherein the indicator portion comprises one or more chemicals configured to activate when exposed to a light source.

6. The IV set of claim 5, wherein the indicator portion is configured to be exposed to the light source upon removing the IV set from light blocking packaging.

7. The IV set of claim 5, the duration monitor device further comprising a cover disposed on the indicator portion, wherein the indicator portion is configured to be exposed to the light source upon removal of the cover from the indicator portion.

8. The IV set of claim 5, wherein the indicator portion is configured to be activated only by exposure to a specific wavelength of light.

9. The IV set of claim 1, wherein the duration monitor device comprises a backing portion and the indicator portion is disposed on the backing portion.

10. The IV set of claim 9, wherein the indicator portion is disposed on a first side of the backing portion, and wherein an adhesive coating is disposed on a second side of the backing portion, the adhesive coating configured to one of affix the duration monitor device to a surface of the IV component, affix the duration monitor device to a tag coupled to the IV component and affix a first portion of the backing portion to a second portion of the backing portion.

11. The IV set of claim 1, wherein the duration monitor device is a portion of material integrally formed with the IV component, and wherein upon exposure of the IV component to an activation source, the remaining portion of the IV component is configured to maintain its color over time and an integrally formed indicator portion is configured to change color after the determined amount of time.

12. The IV set of claim 1, wherein the duration monitor device comprises a first material integrally formed with a second material of a body of the IV component by one of co-injection and co-molding, wherein the body of the IV component is configured to change color after the determined amount of time upon the activation.

13. The IV set of claim 1, wherein the duration monitor device comprises an indicator material disposed on a base material of a body of the IV component by one of coating and over-molding, wherein the body of the IV component is configured to change color after the determined amount of time upon the activation.

14. An intravenous (IV) set, comprising:
an IV component; and
a duration monitor device coupled to the IV component, the duration monitor device comprising a radio frequency identification (RFID) tag configured to be detected by an RFID reader,
wherein the duration monitor is configured to provide an indication of passage of a use-life time period of one of the IV set and the IV component upon being detected by the RFID reader.

15. The IV set of claim 14, wherein the RFID tag is configured to be exposed to the RFID reader upon removing the IV set from RFID shielded packaging.

16. The IV set of claim 14, wherein the RFID tag is configured to be detected by the RFID reader upon being moved within a proximity range of an infusion pump system.

17. A method of operating a duration monitor device, comprising:
coupling the duration monitor device to an intravenous (IV) set component;
activating the duration monitor device upon placing the IV set component into service;
detecting a change in color of an indicator portion of the duration monitor device;
determining a passage of a use-life time period of one of the IV set component and an IV set comprising the IV set component based on the detected change in color of the indicator portion; and
remove the one of the IV set component and the IV set comprising the IV set component from service.

18. The method of claim 17, wherein the indicator portion of the duration monitor device comprises one or more chemicals configured to activate when one of being exposed to humidity in ambient air and being exposed to a light source, the method further comprising one of:
activating the duration monitor device by removing the IV set component from protective packaging; and
activating the duration monitor device by removing a cover removably disposed on the indicator portion.

19. The method of claim 17, wherein the duration monitor device is coupled to the IV set component upon placing the IV set component into service, the method further comprising one of:
removing the duration monitor device from one of a roll and a sheet and placing an adhesive surface of the duration monitor device on a portion of the IV set component, then removing a cover from the indicator portion to activate the duration monitor device;
removing the duration monitor device from a sealed package and placing an adhesive surface of the duration monitor device on a portion of the IV set component, wherein an uncovered indicator portion is activated upon removal of the duration monitor device from the package; and
applying the duration monitor device by an applicator device to place an adhesive surface of the duration monitor device on a portion of the IV set component and to remove a cover from the indicator portion to activate the duration monitor device.

20. The method of claim 17, wherein coupling the duration monitor device to the IV set component comprises one of:
co-injecting an indicator material and a base material to form a body of the IV set component;
co-molding an indicator material and a base material to form a body of the IV set component;
coating an indicator material on a base material of a body of the IV set component; and
over-molding an indicator material onto a base material of a body of the IV set component.
